(19) United States
(12) Patent Application Publication
(10) Pub. No.: US 2002/0086434 A1
(43) Pub. Date: Jul. 4, 2002

(54) Direct determination of acid distributions crudes and crude fractions

(76) Inventors: Stilianos G. Roussis, Brights Grove, CA (US); Lawrence J. Lawlor, Camlachie (CA)

Correspondence Address: EXXONMOBIL RESEARCH AND ENGINEERING COMPANY, P.O. BOX 900, 1545 ROUTE 22 EAST, ANNANDALE, NJ, 08801-0900, US

(21) Appl. No.: 09/957,941

(22) Filed: Sep. 21, 2001

Related U.S. Application Data
(60) Provisional application No. 60/255,659, filed on Dec. 14, 2000.

(51) Int. Cl. G01N 33/22

(52) U.S. Cl. 436/139; 436/173

(57) ABSTRACT

A method for the direct determination of the acid distribution in petroleum crude oil and crude oil fractions by chlorine negative ion chemical ionization mass spectrometry. The crude oil or crude oil fraction is introduced into a mass spectrometer followed by the introduction of a chlorinated reagent capable of producing chloride anions that can react with the acid compounds of the crude oil or crude oil fractions. The mass spectrometer is operated in negative ion mode to selectively detect negatively charged chlorinated adduct ion species. A mass spectra is obtained, from which adduct ions are selected. Peaks from resulting mass chromatograms are identified from which the acid species are quantified.

### CROSS-REFERENCE TO RELATED APPLICATION

This application claims benefit of U.S. provisional patent application serial No. 60/255,659 filed Dec. 14, 2000.

### FIELD OF THE INVENTION

This invention relates to the direct determination of the acid distribution in petroleum crude oil and crude oil fractions by chlorine negative ion chemical ionization mass spectrometry.

### BACKGROUND OF THE INVENTION

It is becoming economically more attractive to process highly acidic crudes because of market constraints. The acid fraction of these crudes creates problems in their transportation and in refining because they are highly corrosive to metals. Typically, the total acid number (TAN) is obtained by ASTM test method D 664 and is used to determine the crude corrosion rate. This test method generates the total amount of the acid content in the crude or crude fraction but does not provide any information about the nature of the acids and their molecular weight distribution. Information regarding the nature of the acids and their molecular weight distribution is often needed to rationalize differences observed in crudes that have similar TAN values, but exhibit dramatically different acid corrosion rates. Non-routine lengthy separation procedures have to be used to extract the acids from the crude for chemical analysis to get this needed information by conventional means. Furthermore, recent studies with high TAN crudes have shown that TAN is not related to corrosivity in a linear fashion but may depend on the nature and the distribution of acids, such as naphthenic acids in the crude.

Naphthenic acids are carboxylic acids having a ring structure, usually five or six-member carbon rings, with side chains of varying length. Such acids are corrosive to metals and must be removed, for example, by treatment with aqueous solutions of alkalis such as sodium hydroxide to form alkali naphthenates. However, the resulting alkali naphthenates become more difficult to separate with increasing molecular weight because they become more soluble in the oil phase as well as becoming more powerful emulsifiers.

It would be advantageous to have a method that would allow the direct characterization and quantification of the acid types in crude oils and fractions since conventional methods of determining TAN do not reveal the nature and concentration of the different naphthenic acid compound types.

### SUMMARY OF THE INVENTION

In accordance with the present invention, there is provided a method for determining the acid distributions in petroleum crude oil and crude oil fractions thereof, which method comprises:

a) introducing said crude oil or crude oil fraction into a mass spectrometer;

b) introducing a chlorinated reagent compound that is capable of producing chloride anions and reacting specifically with acidic compounds in said crude oil or crude oil fraction to form stable, negatively charged chlorinated adduct ion species;

c) operating the mass spectrometer in the negative ion mode to selectively detect negatively charged chlorinated adduct ion species;

d) obtaining a series of mass spectra;

e) selecting, from the mass spectra, adduct ions that are characteristic of the different organic acid species, including those represented by the formula:

CₙH_{2n+z}O₂, where n is the number of carbons, 2n+z is the number of hydrogen atoms and z can take the values: 0 or a negative even integer

f) identifying peaks in the resulting mass chromatograms that are characteristic of the adduct ions; and

g) quantifying the reactive acid species identified by the corresponding adduct ions, wherein the total reactive acid is the weighted sum of the individual reactive acid species.

In a preferred embodiment of the present invention the introduction of the crude oil or crude oil fraction into the mass spectrometer occurs under static conditions.

In another preferred embodiment of the present invention the introduction of the crude oil or crude oil fraction into the mass spectrometer occurs under dynamic flow conditions.

In another preferred embodiment of the present invention, conventional chemical ionization (CI) sources are used for the formation of chlorinated adduct ion species.

In yet another preferred embodiment of the present invention, atmospheric pressure ionization (API) sources are used for the formation of the chlorinated adduct ion species.

### BRIEF DESCRIPTION OF THE FIGURES

FIG. 1 is the chloride ion negative mass spectrum of stearic acid, a model compound.

FIG. 2 is the chloride ion negative mass spectrum of cholanic acid, another model compound.

FIG. 3A is the chloride ion negative mass spectrum of a naphthenic acid extract available from Fluka Inc. and FIG. 3B shows the naphthenic acid distributions of the Fluka acid extract.

FIG. 4A is the chloride ion negative mass spectrum of naphthenic acidic extract available from TCI Inc. and FIG. 4B shows the naphthenic acid distributions of the TCI acidic extract.

FIG. 5A is the chloride ion negative chemical ionization mass spectrum for Heidrun crude acidic extract and FIG. 5B is the chloride ion negative chemical ionization mass spectrum for Heidrun whole crude.

FIGS. 6A and 6B is a comparison of naphthenic acid distributions obtained by chloride ion negative chemical ionization of (A) Heidrun crude acidic extract, and (B) Heidrun whole crude. The total signal for the acids is normalized to 100% molar amount. The z-series of the different naphthenic acid distributions reflect the different acidic compound types (e.g., z=0, fully saturated acids, z=−2, 1-ring naphthenic acids, etc.).

FIGS. 7A and 7B is the chloride ion negative chemical ionization mass spectrum of (A) Bolobo crude acidic extract, and (B) Bolobo whole crude.

FIG. 8 is a comparison of naphthenic acid distributions obtained by chloride ion negative chemical ionization of (A) Bolobo crude acidic extract, and (B) Bolobo whole crude.

FIG. 9 is the chloride ion negative chemical ionization mass spectrum of (A) Bolobo crude acidic extract, and (B) Bolobo crude acidic extract, repeat analysis.

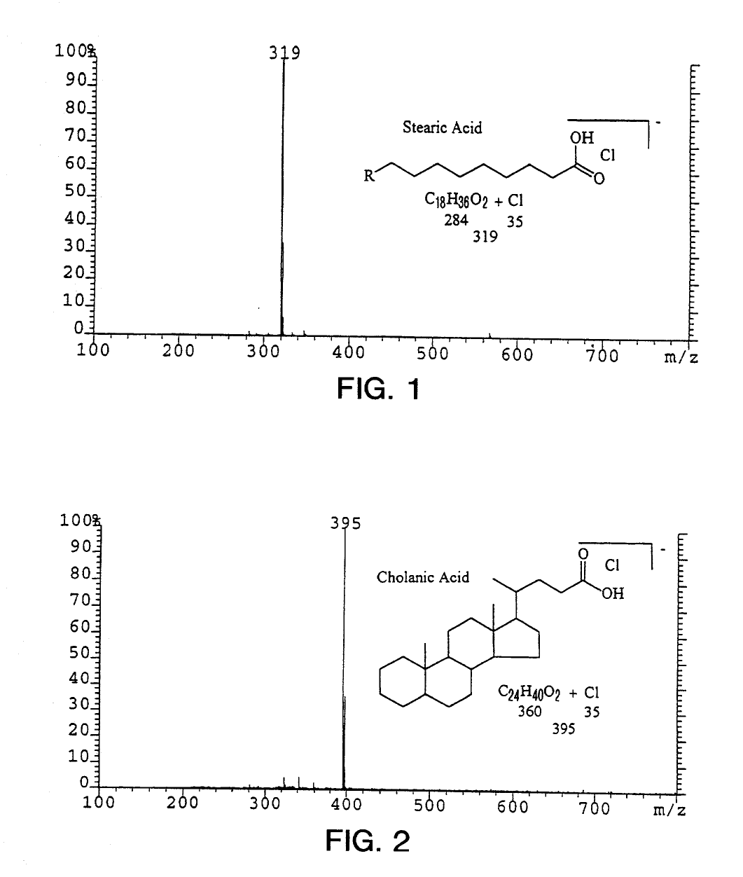

### DETAILED DESCRIPTION OF THE INVENTION

There are several benefits for practicing the present invention. For example, the acids in crudes and crude fractions can be characterized without the need for tedious extractions. This greatly simplifies the long and difficult separations that are conventionally required. Also, critical purchasing and processing decisions involving organic acids can be made by comparison of the content and nature of the acids in difficult crude oils and crude fractions. Further, a fundamental understanding of crude corrosivity and the mechanisms of corrosion in the refinery is gained. This can be done by correlating the content and nature of the acids in different crude oils with crude corrosivity measurements.

The method of the present invention is based on the use of the chloride anion (Cl⁻) as an acid-specific reagent ion for the selective reaction with acidic molecules in petroleum crude samples. The reactions take place in the chemical ionization chamber of a mass spectrometer. On-line analysis of the reaction product ions is achieved by concurrent scanning of the mass spectrometer analyzer. This method is called: chlorine negative ion chemical ionization mass spectrometry (Cl⁻NCI MS). The unique feature of this method is its ability to selectively determine the molecular weight distribution of acidic compounds in petroleum samples without the need for prior extraction of the acidic fractions via time-consuming separation methods. This is due to the selective reaction of free proton-containing acid molecules (e.g. RCOOH) with the chloride anion, in the ionization source of a mass spectrometer, to form a stable negatively charged adduct structure (RCOOHCl⁻), wherein R is one or more paraffinic, naphthenic or aromatic organic groups, or a combination of thereof. A similar reaction does not take place between the chloride anion and the other non-free proton-containing hydrocarbon petroleum molecules. The introduction of the chlorinated reagent compound permits the direct detection and monitoring of the organic acids in crude oils and fraction thereof.

It is taught in Dzidic, I.; Somerville, A. C.; Raia, J. C.; Hart, H. V., Analytical Chemistry, 1988, 60, 1318-1323 that nitrogen trifluoride can be used as a reagent gas for the analysis of naphthenic acids in crudes and waste waters by negative ion chemical ionization mass spectrometry. This technique is based on the formation of a negatively charged RCOO⁻ carboxylate ion and a stable HF molecule. However, this technique is considerably limited for the routine analysis of samples using conventional mass spectrometric instrumentation due to: 1) the need for handling reactive and corrosive fluorine gases, and 2) the use of a specialized ionization source (Townsend discharge ionization source). The chloride ions, as used in the practice of the present invention, unlike fluoride ions, do not lead to the formation of negatively charged RCOO⁻ carboxylate species and a stable HCl molecule. Instead, they form a stable RCOOHCl⁻ adduct ion by simple chloride ion attachment. This is an advantage of the chloride ion method of the present invention. A chemical species with an active proton (i.e. acid) can be easily differentiated based on the observed characteristic isotopic distribution of the chlorinated adduct ion. In the case of the fluoride ion negative chemical ionization experiment, however, the observation of the RCOO⁻ carboxylate ion does not necessarily mean that the chemical species is in its acidic form (RCOOH). It can equally be in the corresponding salt form (e.g. RCOONa, etc.). Additionally, the chloride ion can be easily generated in conventional chemical ionization sources from liquid reagent compounds that are much easier to handle than highly reactive fluorine gases.

The method of the present invention allows for the identification of the “reactive” acid fraction in crude oil as well as a means of monitoring the effects of process options and process parameters on acid distribution and ultimately corrosivity. A continuous series of mass spectra is obtained over a scan range of about 10 to 800 Daltons. The mass spectra data may also be acquired in selected ion monitoring mode. In this mode, care must be taken to select ions representative of the components of interest and to operate under repeatable conditions. A variety of mass spectrometers may be used including low resolution, high resolution, MS/MS, ion cyclotron resonance and time of flight. Low-resolution mass spectrometry is preferred because it is easy to use in the field, although some detailed information may be compromised.

The mass spectrometer is first calibrated in the negative ion mode. This is done in order to detect the negatively charged chlorinated adducts of the organic acids. The mass calibration in the negative ion mode can be done with commercially available mixtures of standard compounds (e.g., perfluorokerosene, etc.) with known masses. These compounds are commercially available calibration mixtures of compounds with known masses that are used to accurately assign the mass scale of the mass spectrum.

Those having ordinary skill in the mass spectrometer art would know how to use the standard calibration mixtures to calibrate the mass scale in either the positive or negative ion mode. Such calibration procedures are conventional and are typically part of the training courses on the use of the instruments.

Chlorinated reagents suitable for use in the present invention are those chlorinated compounds that produce chloride anions and react specifically with acidic compounds in crude oil or crude oil fractions to form stable, negatively charged chlorinated adduct ion species. Chlorinated reagents include chlorinated aliphatic and aromatic compounds such as carbon tetrachloride, chloroform, dichloroethylene, chlorobenzene, dichlorobenzene, benzyl chloride, chloronaphthalene and the like. It is preferred that the chlorinated reagent be one that will produce a single chloride ion and thus result in a single peak in a mass spectrum, instead of a chlorinated compound that produces multiple ions that result in multiple peaks in a mass spectrum. A particularly preferred chlorinated compound is chlorobenzene. Alternatively, a chlorinated reagent compound producing multiple ions, of which one is the chloride ion, can be used, provided that the other ions can be selectively prevented from undergoing reaction with the acidic compounds in the crude oil or crude oil fraction by using mass spectrometers capable of retaining reagent ions of choice. For example, by using an ion trap mass spectrometer it is possible to selectively retain the chloride ion and remove all other ions, thus eliminating possibilities for secondary side reactions between ions other than the chloride ion and the acidic compounds in crude oil or crude oil fractions.

The concentration of the chlorinated reagent compound must be maintained at high enough pressures to achieve chemical ionization in the gas phase of the mass spectrometer. Conventional chemical ionization, or atmospheric pressure ionization mass spectrometric sources can be used for the generation of the chlorinated adduct species. In the case of the conventional chemical ionization sources, the reagent compound can be introduced in a continuous mode via a heated reservoir inlet system or a gas manifold depending on the properties of the chlorinated compound. In the case of the atmospheric pressure chemical ionization and electrospray ionization mass spectrometric sources, a chlorinated solvent can be employed as a mobile phase, or appropriate amounts of a chlorinated reagent compound can be added into a non-chlorinated solvent. The preferred method used here is by injection of approximately 40 μL of reagent compound preferably chlorobenzene) into a heated sample reservoir maintained at about 90° C. Additional reagent compound is injected as needed to maintain the chemical ionization source pressure within the required pressure limits.

Static or dynamic methods of sample introduction can be used. Static methods (e.g., all-glass heated inlet -AGHIS) can be used when chromatographic separation is not required. Dynamic methods such as gas chromatography (GC/MS), liquid chromatography (LC/MS), etc, can provide detailed distributed information about the organic acids. For example, GC/MS can provide the distributions of the acids as a function of boiling point. LC/MS can monitor the organic acids as a function of their polarity. Care should be taken in the selection of the sample introduction method due to the highly reactive nature of the acids so that the acids do not chemically react with the walls or chromatographic columns used to introduce the sample into the mass spectrometer. The direct insertion probe method is preferred. It is a convenient sample introduction method because it permits the volatilization of the acids in the crude oils or their fractions directly into the high vacuum of the mass spectrometer without coming in contact with walls or chromatographic columns.

The constituent crude oil or crude fraction components are introduced into the mass spectrometer to obtain a series of mass spectra. Appropriate mass ranges must be selected to allow for the detection of the entire mass range of interest reflecting the boiling nature of the sample. Scan rates must be selected to permit the acquisition of adequate number of scans for accurate definition of the profiles of peaks when a chromatographic column is used for the separation of compounds. A mass range m/z 10 to 800 and a scan rate of 1 sec/mass decade were the preferred conditions for the experiments.

Classification of the naphthenic acid distributions can be done based on the hydrogen deficiency (z number) of the different acid types. Acid homologues are represented by the general formula: CₙH_{2n+z}O₂ where z specifies the homologous series and n the carbon number of a member compound in the homologous series.

Adduct ions are selected that are characteristic of the different organic acid species. This includes the characterization of the acids according to the chemical formula:

CₙH_{2n+z}O₂

where n is the number of carbons, 2n+z is the number of hydrogen atoms and z can take the values: 0 (aliphatic acids), −2 (1-ring naphthenic acids), −4 (2-ring naphthenic acids), −6 (3-ring naphthenic acids), etc. The number of naphthenic and/or aromatic rings associated with the organic acid is obtained by consideration of the masses of the adduct ions and their chemical formulas. For example, stearic acid has a molecular weight of 284 and the chemical formula is C₁₈H₃₆O₂. The chlorinated negative ion adduct has a mass of 319 (284+35). The observed mass at m/z 319 is the chlorinated negative ion adduct C₁₈H₃₆O₂Cl⁻. Thus, it is possible to calculate the expected mass of the chlorinated adduct ion of an acid compound with a given chemical formula and determine its presence or absence in the mass spectrum. The principles of the same reasoning are used to treat the mass spectrum and assign chemical formulas to the measured masses.

The total acid number (TAN) is obtained from the summation of the total ion current signal of the mass spectra in a crude or crude fraction and comparing it with the signal obtained for a reference crude or fraction with known total acid number.

It will be noted that the instant invention can be used to determine the acid distribution in any liquid medium, both organic as well as aqueous. For example, acid functionalities, including phenols, can be detected in wastewater streams. Furthermore, heteroatoms, such as sulfur, nitrogen and oxygen may be a component of the acid compounds. Also, phenols and other acidic compounds that can form stable chlorinated adduct ions can also be detected by practice of the present invention.

### EXPERIMENTAL PROCEDURE

A JEOL AX505 and a Micromass Zab Spec-OA-TOF sector mass spectrometers were used for these experiments. Crude oil samples were introduced into the ionization source by heating a direct insertion probe from 30° C. to 380° C. at a rate of 32° C./minute. Volatile model compounds and fractions were introduced at a slower heating rate (e.g. 5-10° C./min). The probe temperature was held at the upper temperature limit for 10 minutes. The ionization source temperature was maintained at 200° C. The mass spectrometers were operated in the negative ion chemical ionization mode. The electron kinetic energy was 200 eV and the mass range m/z 33 to 800 was scanned at a scan rate of 1 sec/mass decade.

Chlorobenzene from commercial sources (Aldrich) was used as the reagent compound for chemical ionization. Pressures, typical in chemical ionization experiments with sector instrument ionization sources, were used to produce the negative chemical ionization plasma. The ionization source housing pressure was about 10⁻⁵ Torr. Approximately 40 ml of the reagent compound was introduced into a heated sample reservoir maintained at 90° C. The sample reservoir is interfaced with the ionization source in order to allow the introduction of the reagent compound. The pressure was substantially stable for periods longer than the duration of the experiments (i.e. several hours). Small changes in the ionization source pressure and temperature (about 10 to 15%) did not produce any observable changes in the mass spectra of the reagent compound plasma or the samples.

The chlorobenzene reagent compound produces a single intense Cl⁻ plasma ion peak at m/z 35 with its isotope at m/z 37. The following model acid compounds were used to evaluate the ionization processes using the Cl⁻ plasma: hexanoic acid, 2-ethyl; stearic acid; neo nonadecanoic acid; 1-pyrene butyric acid; and 5-β-cholanic acid. The mass spectra of the model acid compounds were very simple with the most abundant peaks corresponding to the chlorinated acid adduct ions formed by simple chloride ion attachment. This is because the use of chlorobenzene as the reagent instead of a chloride compound such as methylene chloride produces only the chloride plasma ion, which greatly simplifies the network of possible ion-molecule chemical reactions.

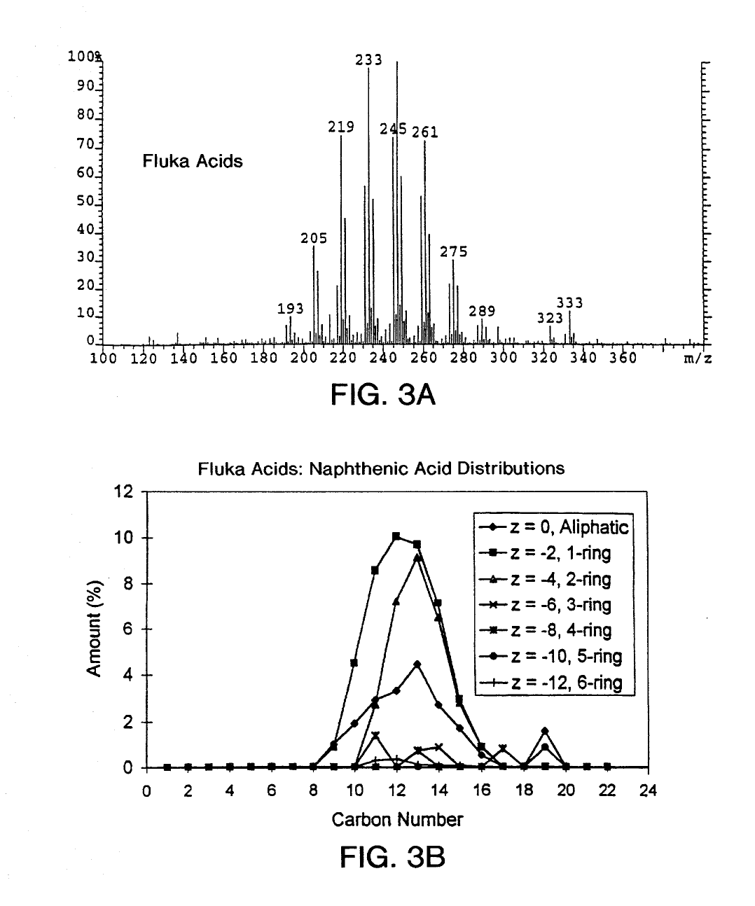

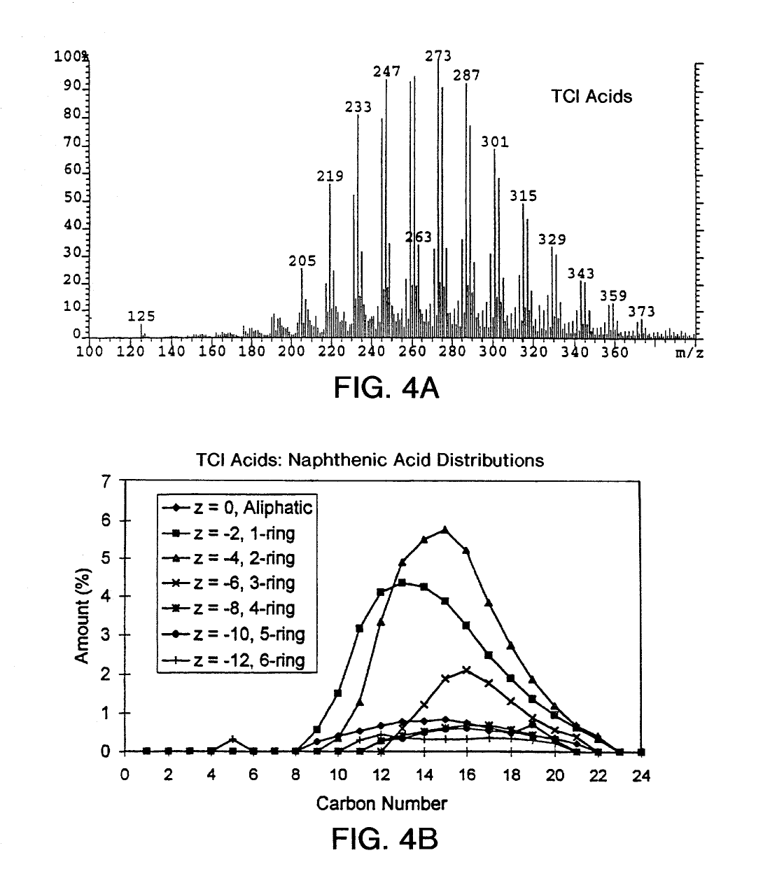

The chloride ion negative chemical ionization mass spectra obtained for two commercially available acidic extracts are given in FIGS. 3 and 4. These are Fluka acidic extract (FIG. 3) and TCI acidic extract (FIG. 4).

FIGS. 3A, and 4A are the chloride ion negative chemical ionization mass spectra and 3B, and 4B show the naphthenic acid distributions for the respective acidic extracts. The total signal for the acids is normalized to 100% molar amount. The z-series of the different naphthenic acid distributions reflect the different acidic compound types (e.g., z=0, fully saturated acids, z=−2, 1-ring naphthenic acids etc. Information with regard to the molecular weight distributions of the acidic extracts can be obtained directly from the mass spectra. For example, the Fluka acids (FIG. 3) have an average molecular weight of approximately 212 (i.e. m/z 247-35 for the C₁₂H₂₃COOH acid). The carbon number distribution ranges from 9 to 19. A computer program was written to treat the raw mass spectra taking into consideration the isotopic abundance of the chlorinated acid adduct ions. The carbon number distributions results given in FIG. 3B are presented in a plot of the relative amount (100%) as a function of acid carbon number. The naphthenic acid distributions are presented using the concept of hydrogen deficiency (z-series). Acid homologues are represented by the general formula CₙH_{2n-z}O₂ where z specifies the homologous series (compound type), and n the carbon number of a member compound in the homologous series. In FIG. 3B −, z=0 corresponds to fully saturated (aliphatic) acid, z=−2 to 1-ring naphthenic acids, z=−4 to 2-ring naphthenic acids, etc. The analysis of the data was restricted to z-series ranging from z=0 to −12 (6-ring naphthenic acids). Equal molar ionization sensitivities were assumed for all compounds.

### DIRECT ANALYSIS OF ACIDS IN CRUDES

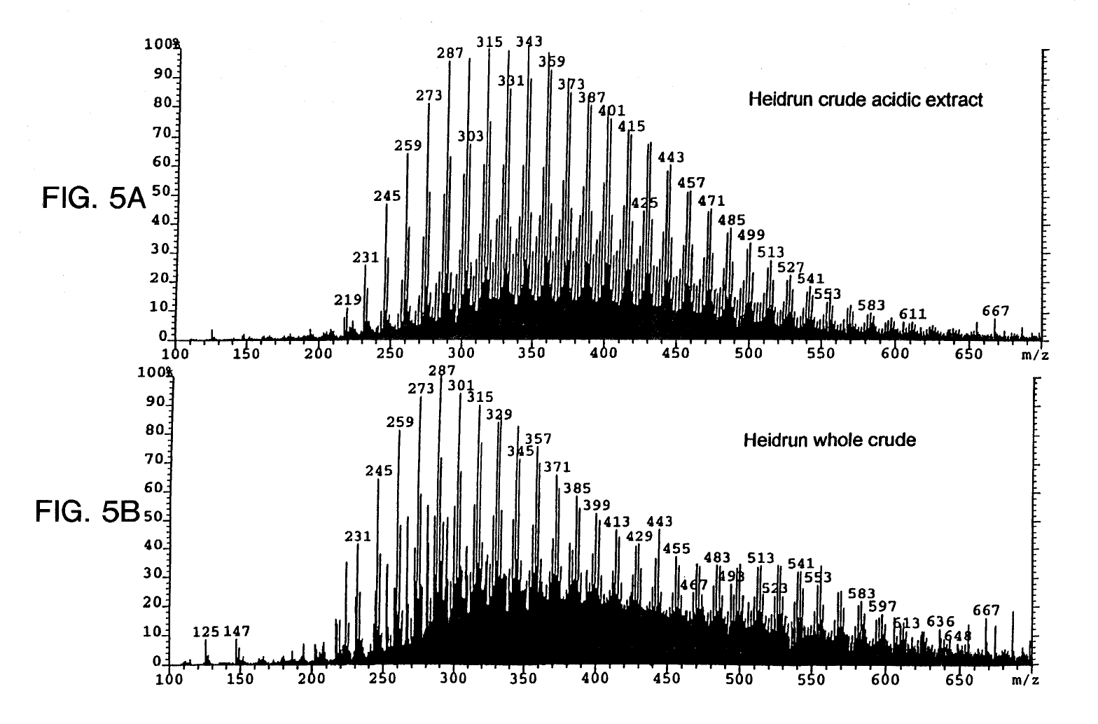

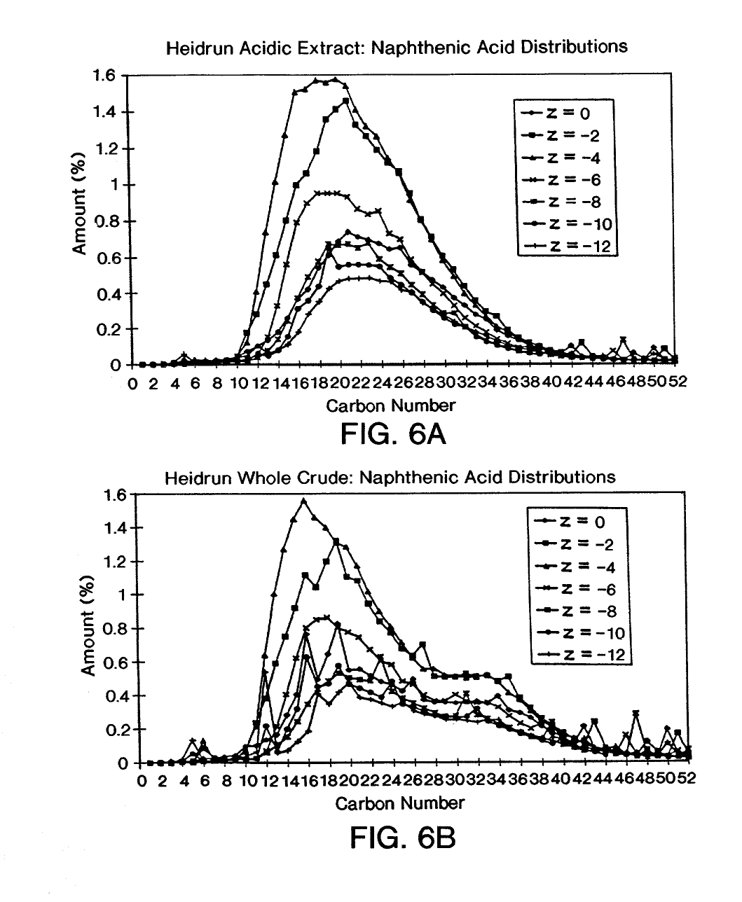

An important feature of chloride ion negative chemical ionization is its capability to selectively analyze structures with acidic protons, in the presence of complex hydrocarbon mixtures. Saturate and aromatic hydrocarbons are not analyzed by the chloride ion negative chemical ionization method. The capability of chloride ion negative chemical ionization for selective analysis of acids in whole crude oils is demonstrated by comparison of the data obtained from the analysis of a set of crude acidic extracts and the corresponding whole crudes. FIG. 5 shows the mass spectra obtained from the analysis of Heidrun crude acidic extract (FIG. 5A) and its corresponding whole crude (FIG. 5B). A very good similarity is obtained between the two mass spectra. The same most abundant ion series is observed for both spectra (m/z 231, 245, 259, 273, etc.). The ion series corresponds to two-ring naphthenic acids (i.e., CₙH₂ₙ₋₄O₂Cl). The corresponding carbon number distributions for the Heidrun acidic extract and the whole crude analyzed by chloride ion negative chemical ionization are shown in FIG. 6 hereof. The results in FIG. 6 clearly show that the chloride ion negative chemical ionization method is highly selective to the analysis of acidic compounds. Similar relative distributions are obtained by the negative chemical ionization method in the analysis of the Heidrun acidic extract and the corresponding whole crude (FIG. 6). The most abundant compound type is due to the 2-ring naphthenic acids, followed by the 1-ring, and 3-ring naphthenic acids.

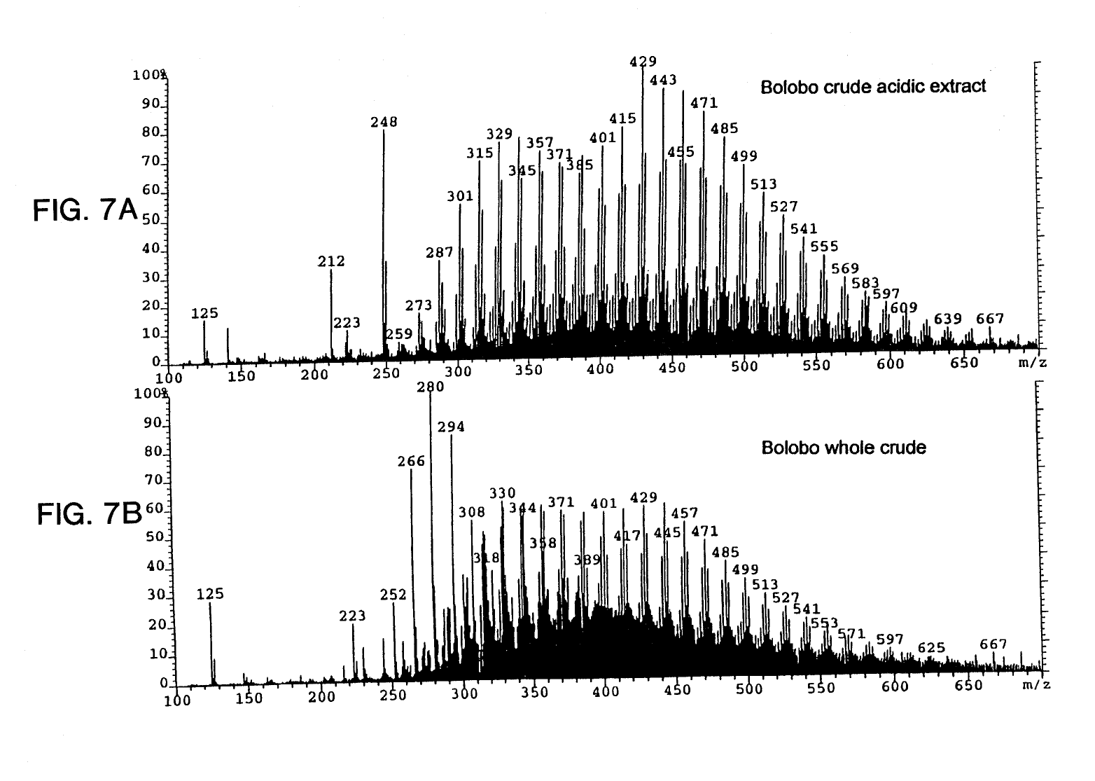

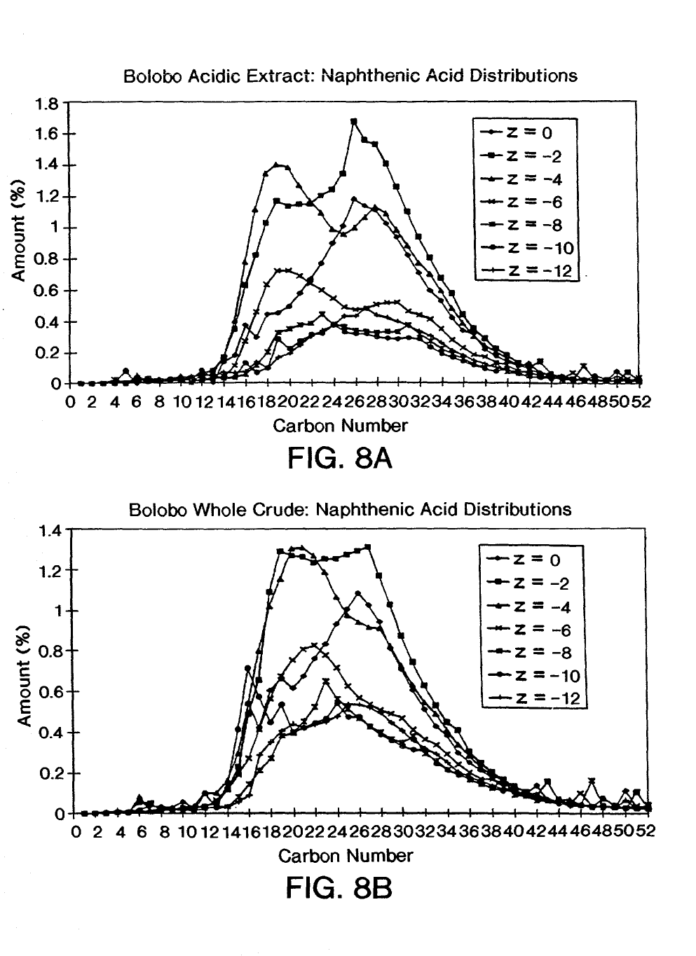

A second example for the selectivity of the chloride ion negative chemical ionization method is given for the analysis of the Bolobo acidic extract and the corresponding whole crude. The mass spectra are shown in FIG. 7 hereof. A very good similarity is obtained for the two mass spectra, indicating an excellent capability of the method to selectively analyze the acids without the need for prior separation of the acids. The similarity is also seen in the naphthenic acid distributions of the two samples shown in FIG. 8. The naphthenic acid distributions for the Bolobo crude are different from those of the Heidrun crude oil (FIG. 8 vs. FIG. 6).

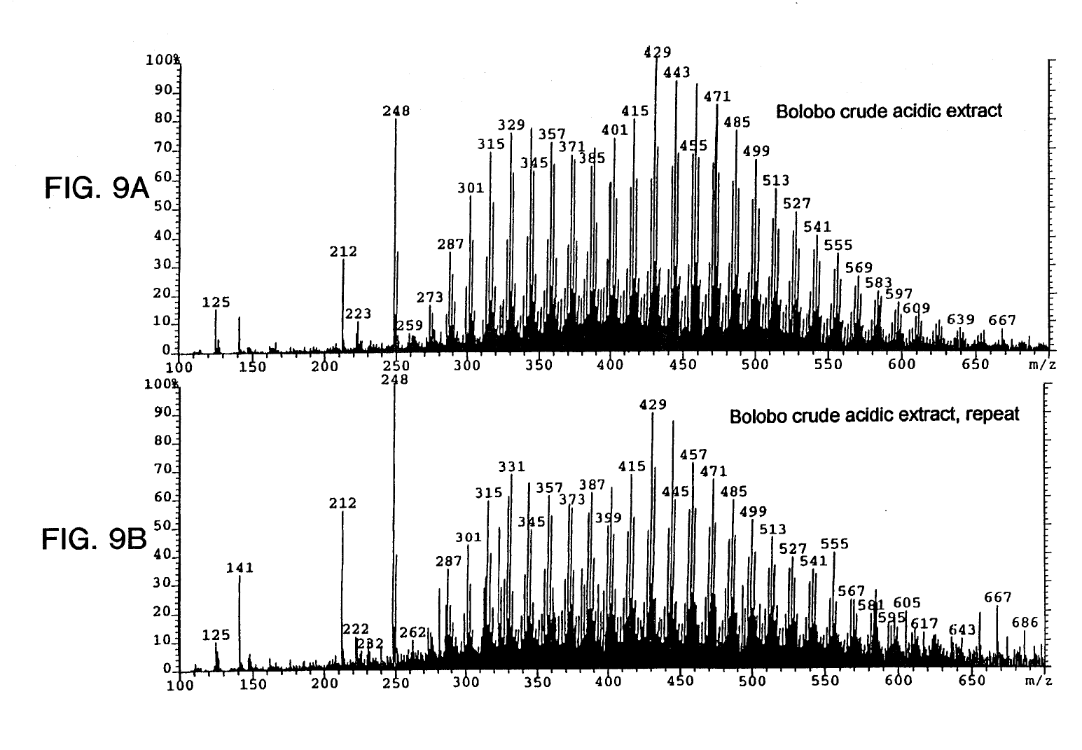

The repeatability of the chloride ion negative chemical ionization method is demonstrated in FIG. 9 which shows the mass spectra obtained from the repeat analysis of the Bolobo crude acidic extract (analysis done within a 5-day interval).

1. A method for determining the acid distributions in petroleum crude oil and crude oil fractions, which method comprises:
a) introducing said crude oil or crude oil fraction into a mass spectrometer;
b) introducing a chlorinated reagent capable of producing chloride anions and reacting specifically with acidic compounds in said crude oil or crude oil fraction to form stable, negatively charged chlorinated adduct ion species;
c) operating the mass spectrometer in the negative ion mode to selectively detect negatively charged chlorinated adduct ion species;
d) obtaining a series of mass spectra;
e) selecting, from the mass spectra, adduct ions that are characteristic of the different organic acid species, including those represented by the formula: CₙH_{2n+z}O₂, where n is the number of carbons, 2n+z is the number of hydrogen atoms and z can take the values 0 or a negative even integer;
f) identifying peaks in the resulting mass chromatograms that are characteristic of the adduct ions; and
g) quantifying the reactive acid species identified by the corresponding adduct ions, wherein the total reactive acid is the weighted sum of the individual reactive acid species.

2. The process of claim 1 wherein said crude oil or crude oil fraction is introduced into said mass spectrometer under static conditions.

3. The process of claim 1 wherein said crude oil or crude oil fraction is introduced into said mass spectrometer under dynamic flow conditions.

4. The process of claim 1 wherein conventional chemical ionization sources are used for the formation of chlorinated adduct ion species.

5. The process of claim 1 wherein atmospheric pressure ionization sources are used for the formation of chlorinated adduct ion species.

6. The process of claim 1 wherein the chlorinated reagent is one that will only produce a single chloride anion that will result in a single peak is said mass spectra.

7. The process of claim 6 wherein the chlorinated reagent is chlorobenzene.

8. A method for determining the naphthenic acid distribution in petroleum crude oil and crude oil fractions, which method comprises:
a) introducing the crude oil or crude oil fraction into a mass spectrometer;
b) introducing a chlorinated reagent compound that is capable of producing chloride anions and reacting specifically with said naphthenic acid compounds represent by RCOOH in the crude or fraction thereof to form stable, negatively charged chlorinated adduct ion species, wherein R is a paraffinic, naphthenic, or aromatic group, or a mixture thereof;
c) operating the mass spectrometer in the negative ion mode to selectively detect negatively charged chlorinated adduct ion species;
d) obtaining a series of mass spectra;
e) selecting, from the mass spectra, adduct ions that are characteristic of the different organic acid species, including those represented by the formula:
CₙH_{2n+z}O_{2,}
where n is the number of carbons, 2n+z is the number of hydrogen atoms and z can be 0 or a negative even integer;
f) identifying peaks in the mass chromatograms that are characteristic of the adduct ions; and
g) quantifying the reactive naphthenic acid species identified by the corresponding adduct ions, wherein the total reactive naphthenic acid species is the weighted sum of the individual acid species.

9. The process of claim 8 wherein said crude oil or crude oil fraction is introduced into said mass spectrometer under static conditions.

10. The process of claim 8 wherein said crude oil or crude oil fraction is introduced into said mass spectrometer under dynamic flow conditions.

11. The process of claim 8 wherein conventional chemical ionization sources are used for the formation of chlorinated adduct ion species.

12. The process of claim 8 wherein atmospheric pressure ionization sources are used for the formation of chlorinated adduct ion species.

13. The process of claim 8 wherein the chlorinated reagent is one that will only produce a single chloride anion that will result in a single peak is said mass spectra.

14. The process of claim 13 wherein the chlorinated reagent is chlorobenzene.
